(19) United States
(12) Patent Application Publication
(10) Pub. No.: US 2015/0173652 A1
(43) Pub. Date: Jun. 25, 2015

(54) TREADMILL ARRANGEMENT AND METHOD FOR OPERATING SAME

(71) Applicant: Zebris Medical GmbH, Isny im Allgäu (DE)

(72) Inventor: Wolfgang Brunner, Isny im Allgau (DE)

(21) Appl. No.: 14/414,107

(22) PCT Filed: May 23, 2013
(86) PCT No.: PCT/EP2013/060600

§ 371 Date: Jan. 11, 2015

(30) Foreign Application Priority Data
Jul. 11, 2012 (DE) 10 2012 212 115.3

(51) Int. Cl. A61B 5/11; A63B 24/00; A61B 5/103; A63B 22/02

(52) U.S. Cl. CPC A61B 5/112; A63B 22/0257; A63B 24/0087; A61B 5/1038

(57) ABSTRACT

A treadmill arrangement, comprising a treadmill frame and an endless belt driven by a drive and running across rollers held in the treadmill frame and whose surface serves as a walking or running surface, with handholds being mounted at upwardly extending side parts or a front part of the treadmill frame on both sides of the belt, and wherein a force sensor system with a holding force evaluation device on the output side for the time-dependent registration and optionally evaluation processing of the holding force, which is subject to the direction in space, introduced by the user into the or each handhold when using the treadmill arrangement and/or actuating means for controlling a parameter and/or a function of the treadmill arranged are assigned to at least one of the handholds.

The invention relates to a treadmill arrangement, comprising a treadmill frame and an endless belt driven by a drive and running across rollers held in the treadmill frame and whose surface serves as a walking or running surface, wherein handholds are mounted at upwardly extending side parts or a front part of the treadmill frame on both sides of the belt. The invention further relates to a method for operating such a treadmill arrangement.

A great product variety of treadmill arrangements used for sportive training purposes have been known for a long time. In the front part (viz, in the direction of use, in front of the user) the frames thereof are usually equipped with a display and operator console for displaying and setting parameters and functions of the arrangement.

Additionally known are apparatus and methods for the gait analysis, which allow the gait to be recorded and analyzed, and which use a treadmill. Reference is here made, for example, to DE 40 27 317 C1 or U.S. Pat. No. 6,010,465 A. A measuring device is described as being known in R. Kram and A. J. Powell: “A treadmill-mounted force platform” Appl. Physiol. 67 (4): 16924698 (1989), wherein a treadmill belt is drawn over a measuring platform or measuring surface, respectively, thereby permitting a continuous detection of forces exerted by a subject's feet on the ground.

Documents EP 2 352 426 and WO 2010/057552 in the name of the applicant describe treadmill systems for use in sports medicine and rehabilitation based on an image display technology, which additionally allows that pressure distribution patterns generated by a person walking on the treadmill are recorded and analyzed synchronously with the displayed images. DE 10 2010 004 504.7 describes a treadmill arrangement which is controlled according to the actual walking behavior of a user, especially a rehabilitation patient, and allows a deactivation process to be triggered.

The invention is based on the object to provide a further improved apparatus of the type described above, which is particularly suited for rehabilitation purposes. It is desired to develop the system further so as to obtain a flexibly usable therapeutic apparatus, by simultaneously enhancing the user acceptance and physiotherapeutic effect.

As far as the product aspects are concerned, this object is achieved by an arrangement according to the features of claim 1, and by a method comprising the features of claim 14 as far as the method aspects are concerned. Useful improvements of the inventive thought are each defined in the dependent claims.

The invention is based on the reflection that walking can be significantly facilitated for elderly people or rehabilitation patients if they make use of walking aids. So-called “wheeled walkers” have become popular in the last years, which need not be specifically positioned by the user, but virtually move along with the user automatically. On the one hand, the invention is thus based on the idea to substantially improve the usability of a treadmill arrangement for rehabilitation purposes by providing the arrangement with corresponding handholds. On the other hand, the invention is based on the idea to provide a force sensor system and/or actuating means for controlling a parameter and/or a function of the treadmill arrangement on at least one of the handholds (preferably on both). Thus, the concept already pursued earlier, inter alia by the applicant, is developed further, namely by providing the treadmill arrangement with a sensor system, respectively, control responsive to the motion behavior of the patient, and gain additional diagnostically relevant data through this sensor system/control and/or influence therapeutically applied parameters, respectively, functions.

In one embodiment of the invention the force sensor system is configured as a multi-axis sensor system for the detection of holding forces of a user subject to the direction in space and/or the actuating means are provided with a multi-axis sensitivity to output control signals subject to the direction of the actuating force. On the one hand, reactions of the user with respect to the treadmill can thus be registered and subjected to a diagnostic evaluation, such as a reflex to hold on to something when the speed increases or an oppositely directed reflex to support oneself against something when the speed is reduced, or also reflexes for compensating an insecure gait. On the other hand, actuation processes can be implemented which are relatively easy to understand and learn, and for the performance of which the user need not give up his/her stable foothold, and which come naturally to him/her from a mental point of view.

More specifically configured is the multi-axis sensor system, respectively, multi-axis sensitivity to distinguish at least between holding forces or actuating forces acting in or opposite to the treadmill moving direction or acting downwardly or upwardly, preferably both. This allows an evaluation both of the aforementioned reflexive reactions to speed changes of the treadmill, respectively, a self-explanatory speed control, but in addition also the detection of a beginning fall or posterior/anterior swaying body movements. For example, the speed of the treadmill can be accelerated by pressing the handholds in a forward direction and reduced by drawing them in a backward direction. Preferably, the multi-axis sensor system, respectively, multi-axis sensitivity is also configured to distinguish between holding forces or actuating forces acting to the left or to the right, so that also lateral swaying body movements can be registered and evaluated.

In another embodiment the force sensor system is combined with the actuating means such that the actuating means are configured to output a control signal in response to a detected force action direction and, optionally, a force amount, and a parameter or a function of the treadmill arrangement can be controlled by the actuating means such that the force applied by the user is reduced or the belt is stopped. Combined with the embodiment explained above, for example, the function is thus obtained, as the treadmill decelerates automatically if the user cannot keep up with the set speed and correspondingly pulls on the handhold, whereas, if the speed of the belt is reduced too much, an instinctive pressing of the handhold may be used to increase the belt speed again. Those skilled in the art will readily appreciate that many other combinations of detection and control functions can be realized in a similar way.

In another embodiment it is provided that the force sensor system and the actuating means are installed together in an individual handhold. If handholds are provided on both sides of the treadmill, preferably each of the two handholds is provided with a combination of force sensors and actuating means. In addition, for controlling at least one parameter or one function of the treadmill arrangement, an additional operating handle or operating lever may be provided in physical allocation to a handhold. An additional operating element of this type may typically serve to trigger a control operation that is deliberately desired by the user (viz. not subject to motion reflexes), e.g. for retrieving selectable functions or illustrations accompanying the training, or an acoustic accompaniment or the like.

In another embodiment of the invention the proposed treadmill arrangement comprises a foot force sensor system for determining a pressure/force distribution on the belt, or a measuring plate located underneath the belt and including a plurality of pressure/force sensors arranged in a matrix-type manner, as well as a foot force evaluation unit connected on the input side to the foot force sensor system, the foot force evaluation unit detecting the position of pressure distribution images of a user walking or running on the belt, and thus the time-dependent positional changes thereof, and a processing unit connected on the output side to the foot force evaluation unit, the processing unit generating gait parameters on the basis of the time/position dependence of the pressure distribution images for characterizing the gait of the user.

In another embodiment it is provided that a gait characterizing stage for deriving a complex gait characteristic of the user, taking into consideration both the parameters, respectively, measured curves determined in the foot force processing unit and the holding force evaluation unit, is provided downstream of the holding force evaluation unit and the foot force processing unit. By this, the treadmill arrangements of the above-mentioned type, provided with the known information possibilities of force measuring plates, can be extended significantly.

In another embodiment the treadmill arrangement is provided with an image display device for the projection of images onto the surface of the belt serving as the walking surface, wherein an image display control device is connected upstream of the image display device, the image display control device being responsive to control signals outputted by the actuating means on the or a handhold. In connection with the image display device, but also without same, a user guide unit may be provided for the visual and/or acoustical output of display-accompanying instructions, in particular by earphones and/or in the form of text insertions.

In another embodiment the modification including a foot force sensor system is provided with a feedback unit which serves to detect contacts of the walking surface by the user at positions at which predetermined picture elements, in particular target placement points, are displayed, or at positions at which no predetermined picture element is displayed, and to output a warning or indicator signal in response thereto. These last-mentioned embodiments serve to extend the possible applications of the treadmill arrangement specifically in the field of rehabilitation, and enhance the user acceptance by allowing a more varied, more demanding and more interesting training.

Method claims for operating, respectively, using the proposed treadmill arrangement substantially ensue directly from the device aspects outlined above and need, therefore, not be explained in more detail.

Advantages and useful features of the invention will be explained in the following description of preferred embodiments by means of the figures. In the figures:

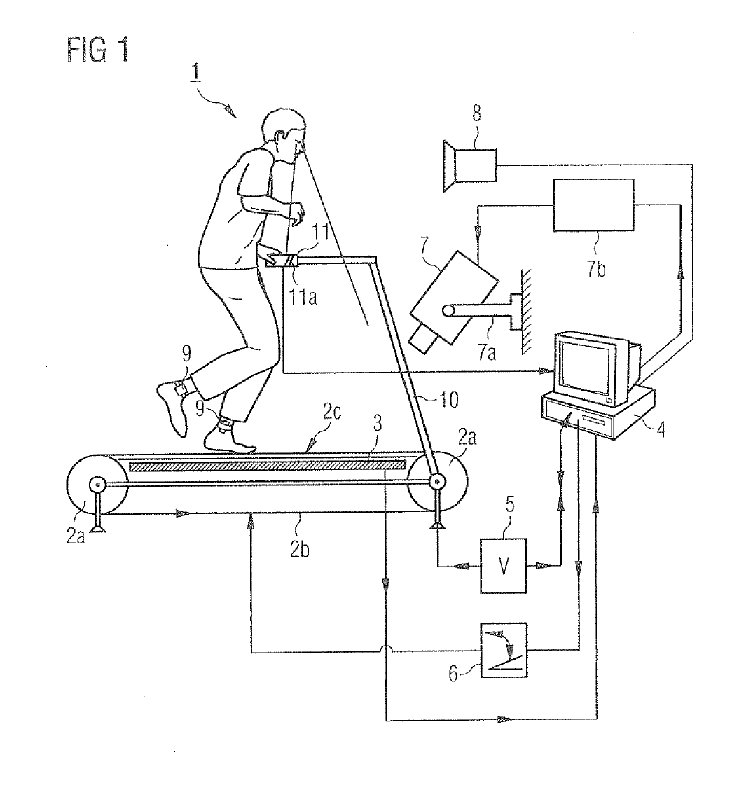

FIG. 1 shows a schematic representation of a first embodiment of the invention,

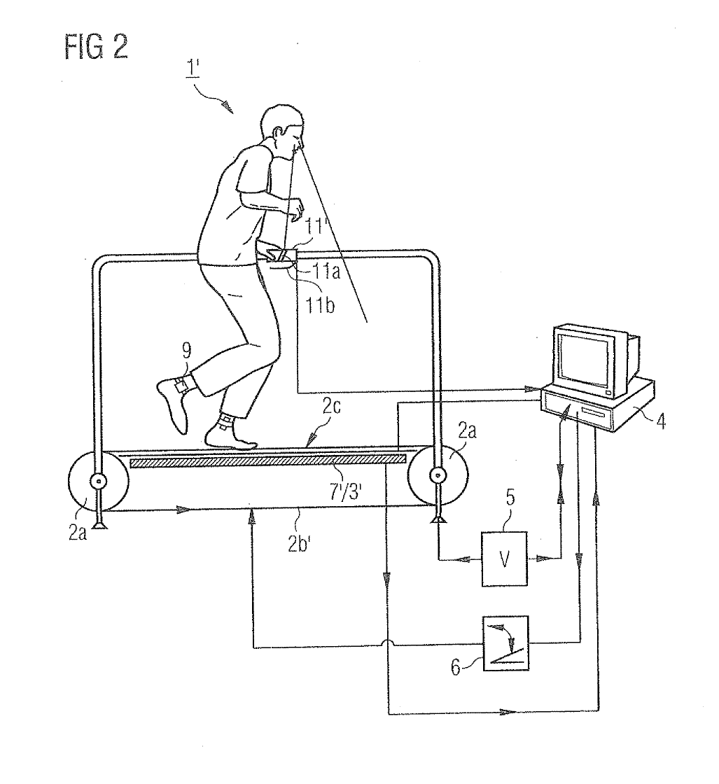

FIG. 2 shows a schematic representation of a second embodiment of the invention,

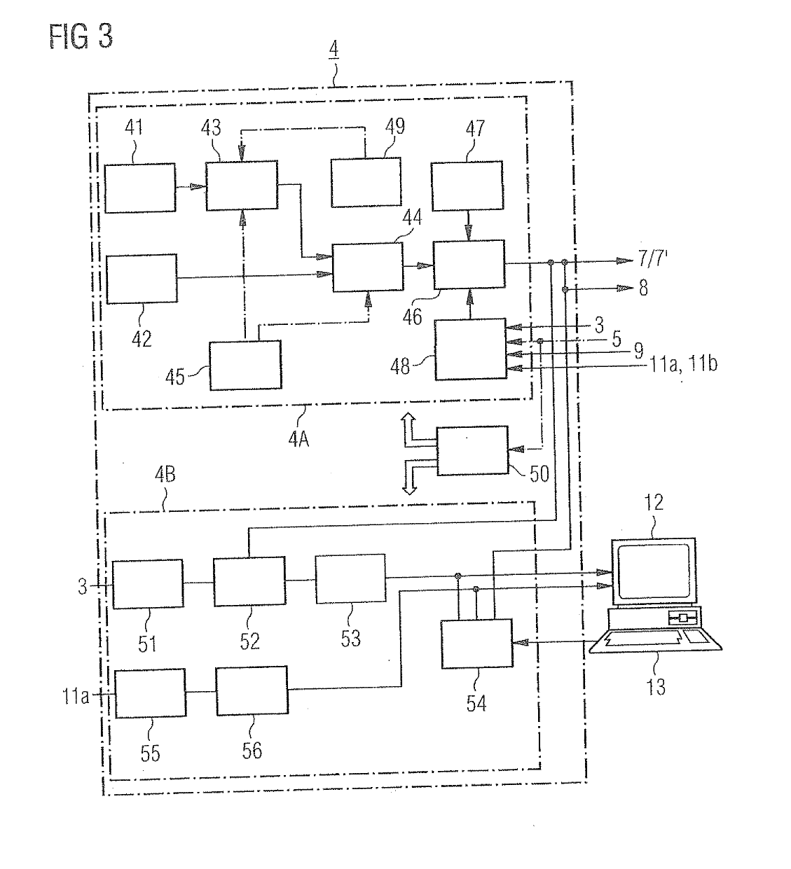

FIG. 3 shows a detailed view of another embodiment,

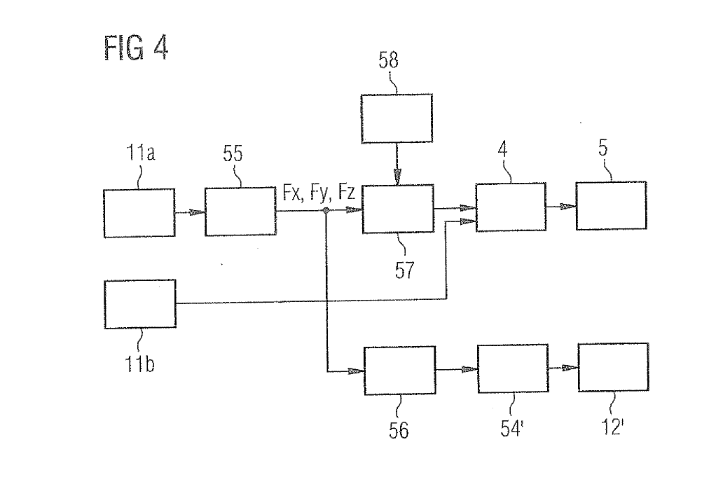

FIG. 4 shows a detailed view of another embodiment, and

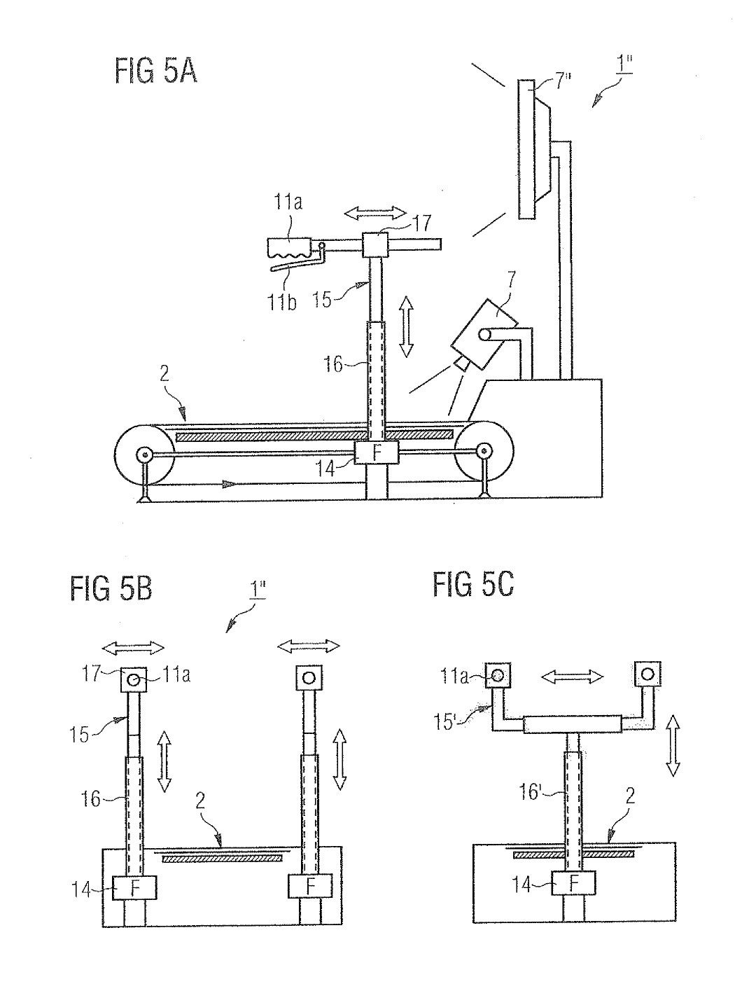

FIG. 5A show cutout illustrations of another embodiment of the invention. to 5C

FIG. 1 shows a treadmill training system I, comprising a treadmill belt 2b running over two rollers 2a, underneath the upper surface of which, which is used by the user as walking surface 2c, a pressure detection plate 3 with a high spatial resolution and having a plurality of (not individually designated) pressure sensors is provided, which pressure sensors are arranged in a matrix-type manner and detect pressure detection images generated by the user as he steps on the treadmill. One of the two rollers 2a is driven and pulls the treadmill belt 2b at a predetermined speed, which is adjusted by a processing and control unit 4 of the arrangement and by a speed controller 5. Moreover, it is possible to adjust an inclination of the treadmill as a whole according to need or, optionally, slightly raise only the front portion thereof by means of a suited inclination actuator 6, which can likewise receive interference signals from the processing and control unit 4 (this is merely symbolically illustrated in the figure). In practice, the inclination of the treadmill may be adjusted, for example, by a hydraulic system lifting the front part or other actuators on the front roller or underneath the walking surface. Also, a plurality of actuators may produce surface irregularities in the walking surface, which may desirable for certain training exercises.

In the embodiment illustrated in FIG. 1, which is strongly simplified, signals characterizing the adjusted speed value of the treadmill are reported back from the speed controller 5 to the processing and control unit 4, where they serve the synchronization of an image displayed on the walking surface 2c by means of a projector (laser Beamer) 7, the image being generated from prestored image elements and/or image sequences (see below).

The image is controlled on the basis of the speed signals in such a way that—especially in connection with another specific embodiment described below—the user is presented an altogether harmonious simulation of a walking environment, preferably combined with the insertion of markings to be touched by the feet and/or with the simulation of obstacles to be climbed over or avoided. Diverging from the representation in the figure, the actual speed of the treadmill can also be detected by a suitable (non-illustrated) sensor system, and the measured value can be supplied to the processing and control unit 4 in order to obtain a (virtually feedback) process control of the image display and synchronized analysis of the pressure distribution patterns.

It is shown in the figure that the projector 7 is fixed to a wall holder 7a in an adjustable manner with respect to angles, so that the direction of the projection encloses with the plane of the treadmill a variable angle. In order to avoid distortions of the images or image elements, which are provided by the processing and control unit 4, due to the acute angle of projection an image signal distortion corrector 7b is connected upstream of the projector 7. This distortion corrector 7b can operate dependent on the actual angular position of the projector 7 in the holder 7a, which is not shown in the figures, however, for reasons of clarity. Moreover, in order to round up the user interface an audio stage 8 is provided (here symbolized as a loudspeaker), by means of which the person doing the workout can receive additional acoustic training instructions. The audio stage 8 can also be realized, for example, bidirectionally in form of a headset, so that the person doing the workout can give an acoustic feedback (e.g. an acknowledgment of received instructions or answers to questions he is asked).

For performing training tasks on the treadmill system it may be of interest to detect the lifting height of the feet from the belt, for example, when the subject is to climb over a virtual obstacle. In another embodiment the subject therefore has a sensor 9 attached to each of his feet, the signals of which can be detected by means of a (non-illustrated) position detection sensor system, which is known per se, so as to draw conclusions on the position or the height of the feet, respectively. Preferably, the sensors are operated time-synchronized with the sensors of the pressure distribution matrix. If appropriate, a precise time synchronization can be generated by means of an infrared or radio signal or by a detection of the moment when the feet contact the belt.

The sensors 9 may be designed as acceleration sensors or mufti-axis acceleration sensors and, if appropriate, are wirelessly connected to the evaluating computer 4. The position of the feet can be calculated from the acceleration signals, especially if the time and position dependence of the pressure distribution patterns can be additionally included in the calculation. In extended arrangements, inertial sensor systems may be employed, in which gyroscopes or sensors for detecting the earth magnetic field are used additionally. Of course, such sensors can also be attached to other body sections, so that the movement of the complete lower extremities or of the whole body can be measured and represented. However, the sensors 9 may also be operated in accordance with other measuring principles, e.g. on the basis of active or passive light markers recorded by stationary cameras, magnetic field sensors, or sensors emitting or receiving ultrasonic waves to or from stationary receivers and determining the position of the feet from the propagation time of the sound. A continuous position determination of the feet of the treadmill user, and specifically a determination of the placement points onto the belt, can basically also be realized by means of a convention (3D) digital camera or a 2D camera.

The pressure sensors of the pressure detection plate may optionally be provided with an analog or—according to a simpler and more inexpensive embodiment—a digital response characteristic (on/off characteristic). Both options are eligible for certain applications, and the system designer will choose one of the options in accordance with the primary use requirements. In a simplified embodiment of the arrangement there may be provided, instead of a pressure detection plate having a plurality of sensors arranged in a matrix, a “rougher” foot force detection by a few force sensors spaced apart from each other underneath the belt or also on the axles of the rollers. On the basis of the measured values obtained by such individual force sensors it is not only possible to determine amount values with interpolation algorithms, but also, to a certain extent, a position dependence of the foot force applied by the user. Below, and in the claims, the term “foot force sensor system” also refers to such a simplified configuration.

A frame 10 of the treadmill arrangement 1, in which the rollers 2a of the treadmill are mounted, includes an upwardly extending front part 9a on which a handhold 11 is respectively attached on both sides of the belt, to which the user holds on in a rehabilitation application of the treadmill arrangement. A multi-axis force sensor system 11a is respectively integrated in the handholds 11, which will be explained in more detail below by means of examples. Output signals of this force sensor system, which allow the detection of the holding forces, respectively, actuating forces applied by the user subject to the direction in space, as well as the signals of the pressure detection plate 3 and the speed controller 5, are transmitted to the evaluating computer 4 for being processed, combined with the signals of the other sensors, in a manner described in more detail below.

FIG. 2 shows a modification of the arrangement illustrated in FIG. 1 and described above. Insofar as the same components of the latter are employed, they are designated with the same reference numbers used in FIG. 1 and will not be explained again below.

The essential modification consists in using a large-surface electro-optical touchscreen 7′ as display device instead of a projector. The upper surface of the touchscreen defines a display surface 2c′ located, in use, underneath the upper section of the endless belt 2b. At the same time, the touchscreen defines a novel pressure detection plate 3′. According to a modified version of the touchscreen principle, and in the light of the actual costs for the arrangement, this combined display/pressure detection device may be replaced by a matrix-type alternating arrangement of a plurality of smaller electro-optical display elements (e.g. LCD displays) and respective adjacent smaller pressure detection plates, or a flexible display screen insensitive to pressure (e.g. of the OLEO type) may be placed over a normal pressure detection plate.

In all cases the endless belt 2b is to be formed of a transparent material, at least in the central portion of its lateral extension, in order to allow the person doing the workout a perception of the images displayed on the display surface 2c′.

In a modification of the frame and handhold configuration, illustrated in FIG. 1 and described above, the treadmill arrangement 1′ comprises a handrail-type treadmill frame 10′ preventing the user (patient) from falling down at the side even if he/her is unable to actively hold on in the event of a sudden feeling of faintness or disorientation. A modified handhold 11′ is provided on the treadmill frame 10′ which comprises, in addition to an integrated force measuring sensor system 11a, an additional actuating lever lib for actively controlling treadmill parameters or functions, e.g. for deliberately decelerating or accelerating, or for causing an inclined position of the belt. Again, the output signals both of the sensor system and the additional actuating lever are transmitted to the evaluating computer 4.

FIG. 3 shows a detailed representation of essential components of the processing and control unit 4 of the arrangement illustrated in FIG. 1 or 2. Not included is here the image signal distortion corrector separately shown in FIG. 1, which is used only in an embodiment of the arrangement where the projector is directed with an inclination to the treadmill.

In a display control part 4a the processing and control unit 4 comprises an image element storing unit 41 and a video memory 42, with an image element mixer 43 and finally a video image element mixer 44 for generating image sequences with predetermined image element insertions being connected downstream thereof. In addition, it is symbolically shown that both mixers 43, 44 can moreover be influenced by control signals of a random generator 45. Also, a display process controller 46 is connected downstream of the second mixer 44, to which is assigned a training program memory 47 and a synchronization unit 48. An image element position controller 49 is connected, in terms of control signals, to the image element mixer 43 and acts on the same so as to vary relative positions of image elements in the ultimate representation.

The synchronization unit 48 can be influenced by signals of the speed controller 5 (not shown in this figure) of the treadmill. Above all, however, the coordinates of actual placement positions of the user's feet are supplied to it on the input side, which (as mentioned above) are derived from the pressure detection images generated by the user's feet on the pressure detection plate 3 according to FIG. 1. Additionally, the signals of sensors 9 and the force sensor system 11a in the handholds and the actuating lever 11b of FIGS. 1 and 2 can furthermore be supplied to the synchronization unit 48 as further input signals, so as to ultimately synchronize the image, specifically with predefined target placement positions for the user's feet, both with the actual speed of the treadmill and the actual placement positions of the user's feet and, if applicable, other motion parameters. With respect to the adaptation of the images presented to the user to his/her current motion sequence the display process controller 46 and the synchronization unit 48 can together be regarded as a display synchronization device of the arrangement.

At the same time, these signals are supplied to a system control unit 50 of the arrangement, which combines and synchronizes the different control procedures of the display and analysis functions and performs necessary adaptations of the data streams and data formats. In the figure this is symbolized by the double arrows pointing to the display control part 4A and the evaluating section 4B.

Moreover, the final image signal provided at the output of the display process controller and, on the other hand, the (space-time-resolved) output signals of the foot force sensor system (pressure distribution plate) 3 are supplied to the evaluating section 4B. The output signal of the pressure distribution plate 3 is freed from interference signals and artefacts in a pressure signal preprocessing stage 51, is synchronized with the image signals in terms of time in a pressure signal time adaptation stage 52 and in terms of space in a pressure signal position adaptation stage 53, and is processed in a training evaluation stage (main processing stage) 53 on the basis of a predetermined training evaluation program. The output signals of the holding force sensor system 11a are preprocessed, in a similar way, in a holding force preprocessing stage 55 for the purpose of an interference elimination, and synchronized in a holding force synchronization stage 56 with the other measured signals, and the results are outputted on a separate display unit 12 of the therapist.

Moreover, they can be processed—together with instructions inputted via an input unit 13 of the therapist—to instructions for the person doing the workout in a user guide stage 54, which instructions are outputted by the display unit 7 or 7′ assigned to the person doing the workout and, optionally, by the audio stage 8 using earphones or loudspeakers.

FIG. 4 shows in a schematic cutout illustration, in the nature of a functional block diagram, essential components, respectively, aspects of the evaluation and control component in another embodiment of the treadmill arrangement. Reference is herein made partly to components and functions shown in FIGS. 1 to 3 and basically explained above.

A commercially available multi-axis force sensor may be used as holding force sensor system 10a, which is suitably connected, with its force detection surface to the parts of a (non-illustrated) handhold, such that the holding, respectively, actuating force applied by the user is introduced into the sensor accurately in terms of direction and amount. On the output side, the holding force sensor system 10a is connected (as already shown in FIG. 3) to a holding force preprocessing stage 55 at the output of which holding force components Fx, Fy and Fz, freed from interferences, are outputted. Again, as shown in FIG. 3a, this preprocessed signal is transmitted via a synchronization stage 56 to a gait characteristic evaluation stage which, in this case (other than in FIG. 3), is not connected to other signal sources and, therefore, is designated with number 54′, and provides a gait characteristic of the user, or essential parameters thereof, determined on the basis of the measured signals of the holding force sensor system 10a, on an evaluating computer 10′ of the doctor or physiotherapist. On the other hand, the preprocessing stage 55 is connected to a holding force comparator unit 57 in which the force, resolved with respect to space and time, is continuously compared with comparative data, respectively, comparative patterns stored in a comparative value memory 58. The comparing process may in particular include a threshold characteristic and, as a result, a first input signal is provided at the output of the comparator unit 57 to the processing and control unit 4 of the treadmill arrangement, which signal, if applicable, is transmitted to the speed controller 5 thereof. Depending on the direction in which the user introduces a force into the handhold, and depending on the amount thereof, speed changes of the treadmill can thus be controlled, if required up to the deactivation (standstill) thereof. This control sequence is largely independent of the user's will and takes into account mainly his/her motion sequence with the use of the handholds.

A second control process is triggered by means of the actuating levers 10b on the handholds. These may in particular comprise a conventional proportional actuator whose output signal is supplied, on the one hand, directly to the processing and control unit 4, and from there to the speed controller 5 where it triggers a speed change of the belt as desired by the user. On the other hand, the signal is also supplied from the actuating lever 10b to the evaluating computer 10′ of the therapist so as to be considered in the registration and evaluation of the gait characteristic of the user.

FIGS. 5A and 5B schematically show in a lateral view, respectively, a cutout illustration, in the nature of a front view, essential parts of another treadmill arrangement 1″ differing from the arrangements 1 according to FIGS. 1 and 1′ of FIG. 2 in substantially two aspects: On the one hand, the treadmill arrangement 1″ comprises two image display devices, namely, in addition to the projection device 7 already shown in FIG. 1, a television screen 7″ which is set up in front of the front end of the treadmill at the height level of a user, and on which additional information can be displayed, e.g. during the training for solving the tasks. This additional image display device, too, is controllable by the sensor system provided on the treadmill, i.e. ultimately subject to the holding and/or foot forces applied by the user.

Moreover, the holding force sensor system, symbolized as a whole as block 14, is placed in the bottom area of the arrangement, at the side of the treadmill, and operatively coupled to the handholds 11a by a multi-axially adjustable lever arrangement 15. In the embodiment according to FIGS. 5A and 5B the lever arrangement 15 has a bar-shaped design and is held in a stand 16 to be vertically displaceable. In addition, a bearing element 17 realizes a longitudinal and lateral adjustability for adaptation to the physical conditions of different users.

FIG. 5C represents a modification of the arrangement described last, in which a modified lever construction 15′ for the force transmission between the handholds 11a and the holding force sensor system 14 is not mounted at the side of the treadmill 2, but in front of same, in a single central stand 16′. In this configuration, one single sensor system component (instead of two according to the embodiments shown in FIGS. 5A and 5B) may be used, and the construction is more simple also from a mechanical point of view, so that costs may be saved. On the other hand, the bar-shaped construction described above may be preferable for certain applications due to the greater mechanical stability thereof and a possibly more sensitive reaction to different holding forces on the left and the right.

The realization of the invention is not limited to the above-described examples, but is also possible in a plurality of modifications within the framework of the competent action of the skilled person.

1. A treadmill arrangement, comprising a treadmill frame and an endless belt driven by a drive and running across rollers held in the treadmill frame and whose surface serves as a walking or running surface, with handholds being mounted at upwardly extending side parts or a front part of the treadmill frame on both sides of the belt, and wherein a force sensor system with a holding force evaluation device on the output side for the time-dependent registration and, optionally, evaluation processing of the holding force, which is subject to the direction in space, introduced by a user into the or each handhold when using the treadmill arrangement and/or actuating means for controlling a parameter and/or a function of the treadmill arrangement are assigned to at least one of the handholds.

2. The treadmill arrangement according to claim 1, wherein the force sensor system is configured as a multi-axis sensor system for the detection of holding forces of a user subject to the direction in space and/or the actuating means are provided with a multi-axis sensitivity to output control signals subject to the direction of the actuating force.

3. The treadmill arrangement according to claim 2, wherein the multi-axis sensor system, respectively, multi-axis sensitivity is configured to distinguish at least between holding forces or actuating forces acting in or opposite to the treadmill moving direction or acting downwardly or upwardly, preferably both.

4. The treadmill arrangement according to claim 3, wherein the multi-axis sensor system, respectively, multi-axis sensitivity is also configured to distinguish between holding forces or actuating forces acting to the left or to the right.

5. The treadmill arrangement according to claim 1, wherein the force sensor system is combined with the actuating means such that the actuating means are configured to output a control signal in response to a detected force action direction and, optionally, a force amount, and a parameter or a function of the treadmill arrangement can be controlled by the actuating means such that the force applied by the user is reduced or the belt is stopped.

6. The treadmill arrangement according to claim 1, wherein the force sensor system and actuating means are arranged on the handhold, in particular installed together in an individual handhold.

7. The treadmill arrangement according to claim 1, wherein an additional operating handle or lever is provided in physical allocation to a handhold to control at least one parameter or a function of the treadmill arrangement.

8. The treadmill arrangement according to claim 1, wherein the force sensor system is connected to the respective handhold by an adjustable lever arrangement for the force transmission.

9. The treadmill arrangement according to claim 1, comprising a foot force sensor system, which includes in particular a plurality of pressure/force sensors arranged in a matrix-type manner for determining a pressure/force distribution on the belt, or a measuring plate located underneath the belt, a foot force evaluation unit connected on the input side to the foot force sensor system, the foot force evaluation unit detecting the position of pressure distribution images of a walking or running user on the belt, and thus the time-dependent positional changes thereof, and a processing unit connected on the output side to the foot force evaluation unit, the processing unit generating gait parameters on the basis of the time/position dependence of the pressure distribution images for characterizing the gait of the user.

10. The treadmill arrangement according to claim 8, wherein a gait characterizing stage for deriving a complex gait characteristic of the user, taking into consideration both the parameters, respectively, measured curves determined in the foot force processing unit and the holding force evaluation unit, is provided downstream of the holding force evaluation unit and the foot force processing unit.

11. The treadmill arrangement according to claim 1, comprising at least one image display device, in particular for the projection of images onto the surface of the belt serving as the walking surface, wherein an image display control device is connected upstream of the or an image display device, the image display control device being responsive to control signals outputted by the actuating means on the or a handhold.

12. The treadmill arrangement according to claim 1, comprising a feedback unit to detect contacts of the walking surface by the user at positions at which predetermined picture elements, in particular target placement points, are displayed, or at positions at which no predetermined picture element is displayed, and to output a warning or indicator signal in response thereto.

13. The treadmill arrangement according to claim 1, comprising a user guide unit for the visual and/or acoustical output of display-accompanying instructions, in particular by earphones or loudspeakers and/or in the form of text insertions by an image display device.

14. A method for operating a treadmill arrangement according to claim 1, in particular for the rehabilitation gait training, wherein the user adopts a position on the belt such that he/her takes hold of the or each handhold, and, at the start of after the start of the belt, output signals of the force sensor system for deriving gait parameters of the user and/or output signals of the actuating means for controlling a parameter and/or a function of the treadmill arrangement are processed.

15. The method according to claim 14, wherein the output signals of the force sensor system and/or the actuating means, combined with output signals of a foot force processing unit for deriving a complex gait characteristic of the user and/or for controlling an image display device and/or user interface of the treadmill arrangement, are processed, in particular for outputting instructions synchronized with the gait of the user.
